(19) United States
(12) Patent Application Publication
(10) Pub. No.: US 2023/0147253 A1
(43) Pub. Date: May 11, 2023

(54) PLATELET PRODUCTION METHOD AND DEVICE

(71) Applicant: KYOTO UNIVERSITY, Kyoto (JP)

(72) Inventors: Koji ETO, Kyoto (JP); Sou NAKAMURA, Kyoto (JP); Shinya SAKUMA, Aichi (JP); Hiroki KUMON, Aichi (JP); Fumihito ARAI, Aichi (JP); Yusuke KASAI, Aichi (JP)

(21) Appl. No.: 17/904,441

(22) PCT Filed: Feb. 18, 2021
(86) PCT No.: PCT/JP2021/006177

§ 371 Date: Aug. 17, 2022

(30) Foreign Application Priority Data
Feb. 20, 2020 (JP) 2020-027289

(51) Int. Cl. C12N 5/078; B01L 3/00

(52) U.S. Cl. CPC C12N 5/0644; B01L 3/502761; B01L 2200/0647; B01L 2300/0816

(57) ABSTRACT

The present invention provides a method for producing a platelet comprising: (a) a step of culturing a megakaryocyte for at least 6 days in a platelet production medium in which a turbulent flow is generated; and (b) a step of injecting the medium comprising the megakaryocyte that has undergone step (a) into a predetermined platelet production device to expose the megakaryocyte to a laminar flow.

### TECHNICAL FIELD

The present invention relates to a platelet production device and to a method for producing a platelet.

### BACKGROUND ART

A platelet preparation is administered to a patient who experiences massive bleeding at the time of surgery or injury, or has a tendency to bleed associated with thrombocytopenia after anticancer agent treatment, for the purpose of treating and preventing a symptom thereof. At present, the production of a platelet preparation depends on blood donation, but there is a need for a stable supply of platelets that is safer in view of infectious disease. To meet this need, currently, a method for producing platelets from megakaryocytes cultured in vitro is being developed. The present inventors have created a method for establishing an immortalized megakaryocyte progenitor cell line (imMKCL) using a pluripotent stem cell as a source.

It is known that by culturing the iPS cell-derived megakaryocyte cell line imMKCL Clone 7 established by the present inventors in a turbulent flow stimulation-dependent vertical stirring culture apparatus (VerMES 8L-culture vessel), human platelets with the same functions as platelets from donated blood can be produced at a level of 100 billion or more, which is the actual amount of blood clinically transfused (see, for example, Non-Patent Document 1 and Patent Document 1).

In addition, a shear stress-dependent microfluidic chip type platelet production bioreactor developed based on the concept that shear stress in the bloodstream is important for platelet production in vivo (see, for example, Non-Patent Documents 2, 3, and 4) is known (see, for example, Patent Document 2).

### CITATION LIST

### Patent Literature

Patent Document 1: WO 2018/164040
Patent Document 2: WO 2017/061528

### Non-Patent Literature

Non-Patent Document 1: Ito Y, Nakamura S, et al., Cell. 2018 Jul. 26; 174(3):636-648
Non-Patent Document 2: Thon, J. N., Mazutis, L., Wu, S., Sylman, J. L., Ehrlicher, A., Machlus, K. R., Feng, Q., Lu, S., Lanza, R., Neeves, K. B., et al. (2014). Platelet bioreactor-on-a-chip. Blood 124, 1857-1867
Non-Patent Document 3: Blin, A., Le Goff, A., Magniez, A., Poirault-Chassac, S., Teste, B., Sicot, G., Nguyen, K. A., Hamdi, F. S., Reyssat, M., and Baruch, D. (2016). Microfluidic model of the platelet-generating organ: beyond bone marrow biomimetics. Sci. Rep. 6, 21700
Non-Patent Document 4: Nakagawa, Y, Nakamura, S., Nakajima, M., Endo, H., Dohda, T., Takayama, N., Nakauchi, H., Arai, F., Fukuda, T., and Eto, K. (2013). Two differential flows in a bioreactor promoted platelet generation from human pluripotent stem cell derived megakaryocytes. Exp. Hematol. 41, 742-748

### SUMMARY OF INVENTION

### Problem to be Solved by the Invention

However, even in the culture method disclosed in Non-Patent Document 1, there has been a problem in that less than 100% of megakaryocytes were observed to produce a platelet within a culture period of 6 days, and that many megakaryocytes do not reach a platelet production form (mode).

In addition, an attempt was made to produce a platelet using the shear stress-dependent microfluidic chip type platelet production bioreactor disclosed in Patent Document 2, but only inferior data were obtained in terms of functionality and efficiency as compared with the production method using the turbulent flow-dependent culture vessel disclosed in Non-Patent Document 1 and Patent Document 1.

There is a need for a method for producing a platelet having sufficient functionality with high efficiency by fully maturing a megakaryocyte into a platelet-producing form, and for an apparatus capable of achieving this.

### Means for Solving the Problems

The present inventors have earnestly researched and, as a result, found that by carrying out turbulent flow-dependent culture of a megakaryocyte for a predetermined period of time and then subjecting a medium containing the megakaryocyte to a shear stress-dependent microfluidic chip type platelet production bioreactor, a platelet having sufficient functionality can be produced with high efficiency, and they have thus completed the present invention.

That is, the present invention includes the following aspects.
[1] A method for producing a platelet, including the steps of:
(a) culturing a megakaryocyte for at least 6 days in a platelet production medium in which a turbulent flow is generated; and
(b) injecting the medium including the megakaryocyte that has undergone step (a) into a platelet production device to expose the megakaryocyte to a laminar flow,

in which the platelet production device includes

an injection port for a megakaryocyte,

a platelet collection section, and

a channel extending from the injection port to the collection section, the channel is configured such that

a height of an end of the channel on the injection port side is greater than a maximum diameter of a megakaryocyte to be injected,

a height of an end of the channel on the collection section side is less than a minimum diameter of a megakaryocyte to be injected and greater than a maximum diameter of a platelet, and

the height of the channel decreases from the injection port toward the collection section, and thereby, the platelet production device is configured to make it possible to expose the megakaryocyte to the laminar flow in a state in which the megakaryocyte is captured in the channel, and to make it possible to release a platelet produced by the megakaryocyte from the channel into the collection section.
[2] The method according to [1], in which a width of the channel changes from the injection port toward the collection section, and the change correlates with a diameter distribution of the megakaryocyte to be injected.
[3] The method according to [2], in which when a distance of the channel from the end portion on the injection port side is x, a height of the channel at the distance x is h(x), a width of the channel at the distance x is w(x), and a diameter of the megakaryocyte is x_{d},
w(x) is determined according to a frequency of a megakaryocyte having a diameter of h(x), and the channel is configured such that w(x) increases as a frequency of a megakaryocyte having a diameter x_{d} of h(x) increases.
[4] The method according to any one of [1] to [3], in which the platelet production device includes a plurality of pillars rising from a bottom surface of the end portion in the channel on the collection section side.
[5] The method according to any one of [1] to [4], in which the method includes, before the step of culturing a megakaryocyte, a step of forcibly expressing an oncogene, a polycomb gene, and an apoptosis suppressor gene in a cell more undifferentiated than a megakaryocyte to obtain an immortalized megakaryocyte.
[6] The method according to any one of [1] to [5], in which the method includes a step of collecting the platelet from the collection section of the platelet production device.
[7] The method according to any one of [1] to [6], in which the step of culturing a megakaryocyte for at least 6 days is carried out using a shaking flask or a culture vessel including an unsteadily operable blade.
[8] A platelet production device including:

an injection port for a megakaryocyte;

a platelet collection section; and

a channel extending from the injection port to the collection section, in which the channel is configured such that

a height of an end of the channel on the injection port side is greater than a maximum diameter of a megakaryocyte to be injected,

a height of an end of the channel on the collection section side is less than a minimum diameter of a megakaryocyte injected and greater than a maximum diameter of a platelet, and

the height of the channel decreases from the injection port toward the collection section, and thereby, the platelet production device is configured to make it possible to expose the megakaryocyte to a laminar flow in a state in which the megakaryocyte is captured in the channel, and make it possible to release a platelet produced by the megakaryocyte from the channel into the collection section.
[9] The device according to [8], in which when a distance of the channel from the end portion on the injection port side is x, a height of the channel at the distance x is h(x), a width of the channel at the distance x is w(x), and a diameter of the megakaryocyte is x_{d},
w(x) is determined according to a frequency of a megakaryocyte having a diameter of h(x), and the channel is configured such that w(x) increases as a frequency of a megakaryocyte having a diameter x_{d} of h(x) increases.

### Advantageous Effects of Invention

According to the method for producing a platelet according to the present invention, it is possible to efficiently produce a human platelet exhibiting a function equivalent to that of a platelet from donated blood. In addition, the platelet production device of the present invention has a predetermined feature in the width of the channel, and thus, even after injecting a megakaryocyte and capturing the megakaryocyte in the channel, the constant state of liquid flow into the device can be maintained. Because of this, it is possible to apply a constant shear stress to the megakaryocyte, and it is possible to reduce the variation in the number of platelets produced to effectively produce platelets.

### BRIEF DESCRIPTION OF DRAWINGS

FIG. 1 is a diagram schematically showing the method for producing a platelet according to one embodiment of the present invention.

FIG. 2 is a conceptual cross-sectional view showing one example of a culture vessel for a megakaryocyte, which is preferably used in the method for producing a platelet according to one embodiment of the present invention.

FIG. 3 is a plan view of the culture vessel shown in FIG. 2.

FIG. 4 is a conceptual perspective view showing one example of the platelet production device according to a second aspect of the present invention.

FIG. 5A is a diagram illustrating variables in a design of the channel of the platelet production device according to the second aspect of the present invention.

FIG. 5B is a graph showing one example of a diameter distribution in a megakaryocyte cell population.

FIG. 5C is a graph showing one example of designs of the channel h(x) and the distance x from the end portion on the injection port side of the channel of the platelet production device according to the second aspect of the present invention.

FIG. 5D is a graph showing one example of designs of the channel width w(x) and the distance x from the end portion on the injection port side of the channel of the platelet production device according to the second aspect of the present invention.

FIG. 6 is a conceptual cross-sectional view showing a production example of the platelet production device according to the second aspect of the present invention.

FIG. 7 is a graph showing the numbers of CD41a/CD42b positive platelets produced when megakaryocytes on days 5, 6, 7, and 8, of a Gene OFF maturation culture were introduced into a platelet production device to produce platelets.

FIG. 8 is a graph showing results obtained by introducing megakaryocytes on days 5, 6, 7, and 8, of Gene OFF maturation culture into a platelet production device to produce platelets, collecting a platelet mixed culture solution, and measuring the platelet hemostatic function (PAC-1).

FIG. 9 is a graph showing results obtained by introducing megakaryocytes on days 5, 6, 7, and 8, of a Gene OFF maturation culture into a platelet production device to produce platelets, collecting a platelet mixed culture solution, and measuring Annexin V, which is a platelet aging marker.

FIG. 10 is FACS diagrams showing Annexin V measurement results.

### EMBODIMENT FOR CARRYING OUT THE INVENTION

Hereinafter, embodiments of the present invention will be described. However, the present invention is not limited to the embodiments described below.

### 1. Spherical Culture

The present invention, according to one embodiment, relates to a method for producing a platelet. The method for producing a platelet includes at least the following steps:
(a) a step of culturing a megakaryocyte for at least 6 days in a platelet production medium in which a turbulent flow is generated; and
(b) a step of injecting the medium containing the megakaryocyte that has undergone step (a) into a platelet production device to expose the megakaryocyte to a laminar flow.

In the method for producing a platelet according to the present invention, the megakaryocyte to be cultured in step (a) above refers to a megakaryocyte defined below. The term “megakaryocyte” is the largest cell present in the bone marrow in vivo and is characterized by releasing a platelet. A megakaryocyte is characterized by being positive for cell surface markers CD41a, CD42a, and CD42b, and may also further express at least one marker selected from the group consisting of CD9, CD61, CD62p, CD42c, CD42d, CD49f, CD51, CD110, CD122, CD131, and CD203c. A “megakaryocyte” has a genome that is 16 to 32 times greater than that of a normal cell when it is multinucleated (polyploidized), but as used herein, “megakaryocyte” simply refers to both a multinucleated megakaryocyte and to a megakaryocyte before multinucleation, as long as it has the above characteristics. The “megakaryocyte before multinucleation” is also synonymous with “immature megakaryocyte” or “proliferative megakaryocyte.” The “megakaryocyte before multinucleation” means, for example, a cell that is more undifferentiated than a multinucleated megakaryocyte and a mononuclear or binuclear cell that is CD41a positive, CD42a positive, and CD42b positive, and has not yet undergone nuclear polyploidization. The megakaryocyte before multinucleation can be obtained by various known methods, and for example, may be obtained by isolation from bone marrow, umbilical cord blood, and peripheral blood, or by differentiation induction from a pluripotent stem cell such as an ES cell or an iPS cell. The megakaryocyte can be obtained by various known methods, which are not particularly limited, and may be a megakaryocyte obtained from any source by any method. For example, the megakaryocyte may also be obtained by further differentiation induction of the above megakaryocyte before multinucleation. In addition, as used herein, the “megakaryocyte” simply referred to may refer not only to a single megakaryocyte, but also to a megakaryocyte population composed of a plurality of megakaryocytes. The megakaryocyte cell population is generally a population composed of heterogeneous cells having a predetermined distribution in diameter.

In a certain embodiment, the method for producing a platelet according to the present invention includes, before step (a), a step of forcibly expressing an oncogene, a polycomb gene, and an apoptosis suppressor gene in a cell more undifferentiated than a megakaryocyte to obtain an immortalized megakaryocyte.

Non-limiting examples of such a method for producing an immortalized megakaryocyte include the method disclosed in WO 2011/034073. In this method, an immortalized megakaryocyte cell line that proliferates indefinitely can be obtained by forcibly expressing an oncogene and a polycomb gene in a “cell more undifferentiated than a megakaryocyte.” In addition, an immortalized megakaryocyte cell line can also be obtained by forcibly expressing an apoptosis suppressor gene in a “cell more undifferentiated than a megakaryocyte” according to the method disclosed in WO 2012/157586. By turning off the forced expression of the genes, these immortalized megakaryocyte cell lines become multinucleated and start to release a platelet. Therefore, the step of culturing in the present invention can also be said to be a step of turning off the forced expression of the genes and culturing.

In the step of obtaining an immortalized megakaryocyte, which can be carried out before step (a), the methods disclosed in the above documents may be combined in order to obtain the megakaryocyte. In that case, an oncogene, a polycomb gene, and an apoptosis suppressor gene may be forcibly expressed at the same time or sequentially. For example, a multinucleated megakaryocyte may be obtained by forcibly expressing an oncogene and a polycomb gene and suppressing this forced expression, and then forcibly expressing an apoptosis suppressor gene and suppressing this forced expression. In addition, a multinucleated megakaryocyte can also be obtained by simultaneously forcibly expressing an oncogene, a polycomb gene, and an apoptosis suppressor gene and simultaneously suppressing this forced expression. A multinucleated megakaryocyte can also be obtained by first forcibly expressing an oncogene and a polycomb gene, then forcibly expressing an apoptosis suppressor gene, and simultaneously suppressing these forcible expressions. As used herein, the step of forcibly expressing a gene may be referred to as a proliferation culture step, a proliferation phase, or a proliferation-capable state, and the step of suppressing the forced expression may be referred to as a maturation culture step or a maturation phase.

As used herein, “cell more undifferentiated than a megakaryocyte” means a cell having a capability of differentiating into a megakaryocyte and at any of various stages of differentiation ranging from a hematopoietic stem cell lineage to a megakaryocyte. Non-limiting examples of cells more undifferentiated than a megakaryocyte include a hematopoietic stem cell, a hematopoietic progenitor cell, a CD34 positive cell, and a megakaryocyte-erythroid progenitor cell (MEP). These cells can also be obtained by isolation from, for example, bone marrow, umbilical cord blood, or peripheral blood, or by differentiation induction from a pluripotent stem cell such as an ES cell or an iPS cell, which is a more undifferentiated cell.

As used herein, the term “oncogene” refers to a gene that induces canceration of a cell in vivo, and examples thereof include an MYC family gene (for example, c-MYC, N-MYC, or L-MYC), an SRC family gene, an RAS family gene, an RAF family gene, and a protein kinase family gene such as c-Kit, PDGFR, or Abl.

The term “polycomb gene” is known as a gene that negatively regulates the CDKN2a (INK4a/ARF) gene and functions to avoid cell aging (Ogura et al., Regenerative Therapy vol. 6, No. 4, pp. 26-32; Jesus et al., Nature Reviews Molecular Cell Biology vol. 7, pp. 667-677, 2006; Proc. Natl. Acad. Sci. USA vol. 100, pp. 211-216, 2003). Non-limiting examples of the polycomb gene include BMI1, Mel18, Ring1a/b, Phc1/2/3, Cbx2/4/6/7/8, Ezh2, Eed, Suz12, HDAC, and Dnmt1/3a/3b.

The term “apoptosis suppressor gene” refers to a gene having the function of suppressing cell apoptosis, and examples thereof include BCL2 gene, BCL-xL gene, Survivin gene, and MCL1 gene.

Forced expression of a gene and turning off of the forced expression can be carried out by the method disclosed in WO 2011/034073, WO 2012/157586, WO 2014/123242, or Nakamura S., et al., Cell Stem Cell. 14, 535-548, 2014, other known methods, or a method equivalent thereto. For example, when an agent-responsive gene expression induction system such as a Tet-on (registered trademark) or Tet-off (registered trademark) system is used for forced expression of a gene and turning off thereof, in the step of forcing expressing, the forced expression may be suppressed (turned off) by adding the corresponding agent, for example tetracycline or doxycycline, in a medium and removing the agent from the medium.

The culture conditions for a megakaryocyte when forcibly expressing a gene and suppressing (turning off) the forced expression can be normal conditions. For example, the temperature can be about 35° C. to about 42° C., about 36° C. to about 40° C., or about 37° C. to about 39° C., and 5 to 15% CO₂ and/or 20% O₂ may be used.

Specifically, the step of forcibly expressing the above genes in a cell more undifferentiated than a megakaryocyte can be carried out according to a conventional method used by those skilled in the art, and for example, the step can be carried out by transfecting these genes into a cell more undifferentiated than a megakaryocyte in the form of a vector expressing these genes or a protein or RNA encoding these genes. Furthermore, the step can be carried out by contacting a low molecular weight compound or the like that induces the expression of these genes with a cell more undifferentiated than a megakaryocyte.

Examples of the vector expressing these genes that can be used include a viral vector such as a retrovirus, lentivirus, adenovirus, adeno-associated virus, herpesvirus, and Sendai virus, and an animal cell expression plasmid (for example, pA1-11, pXT1, pRc/CMV, pRc/RSV, or pcDNAI/Neo). A retroviral vector or a lentiviral vector can be preferably used in that the expression can be carried out by a single transfection. Examples of a promoter used in an expression vector include an EF-a promoter, a CAG promoter, an SRa promoter, an SV40 promoter, an LTR promoter, a CMV (cytomegalovirus) promoter, an RSV (Rous Sarcoma Virus) promoter, MoMuLV (Moloney Murine Leukemia Virus) LTR, and an HSV-TK (herpes simplex virus thymidine kinase) promoter. The expression vector may contain, if desired, an enhancer, a poly-A addition signal, a selection marker gene, an SV40 origin of replication, or the like, in addition to the promoter. Examples of the selection marker gene that is useful include a dihydrofolate reductase gene, a neomycin resistance gene, and a puromycin resistance gene.

An agent-responsive vector may be used as the above expression vector. For example, in order to control the expression of the gene using tetracycline or doxycycline, an agent-responsive vector having a tetracycline-reactive element in the promoter region may be used. In addition thereto, in order to excise the gene from the vector using a Cre-loxP system, an expression vector in which loxP sequences are disposed in such a way as to flank the gene, the promoter region, or both thereof between the loxP sequences may be used.

In the production of a megakaryocyte, at least one of (i) a step of treating a cell being cultured after forcibly expressing an apoptosis suppressor gene therein, using an actomyosin complex function inhibitor, and (ii) a step of treating the same using a ROCK inhibitor may be included. These treatments can promote more stable proliferation and multinucleation.

Those skilled in the art can determine the optimum concentration of an actomyosin complex function inhibitor, a ROCK inhibitor, or the like when treating a cell therewith, in advance by preliminary experimentation. In addition, those skilled in the art can also appropriately select the period of time, method, and the like for the treatment. For example, in the case of treatment using blebbistatin, which is a myosin heavy chain II ATPase inhibitor, about 2 to 15 μg/ml or 5 to 10 μg/ml is added to a culture solution, and the culture period is, for example, about 5 to 10 days and particularly preferably about 6 to 7 days. In addition, Y27632, which is a ROCK inhibitor, can be used at about 5 to 15 μM or 8 to 12 μM, and preferably about 10 μM. The treatment time of Y27632 is about 10 to 21 days and preferably about 14 days.

Examples of the ROCK (Rho-associated coiled-coil forming kinase/Rho-binding kinase) inhibitor include [(R)-(+)-trans-N-(4-pyridyl)-4-(1-aminoethyl)-cyclohexanecarboxamide.2HCl.H₂O](Y27632). In some cases, an antibody or a nucleic acid (for example, shRNA) that inhibits Rho-kinase activity can also be used as the ROCK inhibitor.

After the step of forcibly expressing the genes, a step of culturing the megakaryocyte or megakaryocyte progenitor cell obtained in the step of forcibly expressing the genes, in a platelet production medium, is carried out. A method for suppressing or silencing the forced expression in the step of culturing may also be achieved, for example, when the forced expression is carried out using an agent-responsive vector in the preceding step, by not contacting the corresponding agent with the cell. Specifically, when forced expression of a gene is carried out using doxycycline or tetracycline, the forced expression can be suppressed by culturing the cell in a medium from which such an agent has been removed. In addition thereto, when the above vector including LoxP is used, the suppression may also be achieved by transfecting Cre recombinase into the cell. Furthermore, when a transient expression vector and RNA or protein transfection are used, the suppression may also be achieved by stopping the contact with the vector or the like. The medium used in this step can be carried out using the same medium as described above.

The platelet production medium used in step (a) is not particularly limited, and a known medium suitable for producing a platelet from a megakaryocyte or a medium equivalent thereto can be appropriately used. For example, a medium used for culturing an animal cell can be prepared as a basal medium. Examples of the basal medium include IMDM medium, Medium 199 medium, Eagle's Minimum Essential Medium (EMEM) medium, αMEM medium, Dulbecco's modified Eagle's Medium (DMEM) medium, Ham's F12 medium, RPMI 1640 medium, Fischer's medium, Neurobasal Medium (Life Technologies), and a mixed medium thereof.

The medium may contain serum or plasma, or may be serum-free. The medium can also contain, as needed, one or more substances such as albumin, insulin, transferrin, selenium, a fatty acid, a trace element, 2-mercaptoethanol, thiolglycerol, monothioglycerol (MTG), a lipid, an amino acid (for example, L-glutamine), ascorbic acid, heparin, a non-essential amino acid, a vitamin, a growth factor, a low molecular weight compound, an antibiotic, an antioxidant, pyruvic acid, a buffer, an inorganic salt, or a cytokine. The cytokine is a protein that promotes hematopoietic differentiation, and examples thereof include vascular endothelial growth factor (VEGF), thrombopoietin (TPO), various TPO-like agents, Stem Cell Factor (SCF), ITS (insulin-transferrin-selenite) supplement and an ADAM inhibitor. The preferable medium in the present invention is IMDM medium containing serum, insulin, transferrin, selenium, thiolglycerol, ascorbic acid, and TPO. This IMDM medium may further contain SCF and may further contain heparin. The concentration of each substance is not particularly limited, and for example, the concentration of TPO may be about 10 ng/mL to about 200 ng/mL, or about 50 ng/mL to about 100 ng/mL, the concentration of SCF can be about 10 ng/mL to about 200 ng/mL or about 50 ng/mL, and the concentration of heparin can be about 10 U/mL to about 100 U/mL or about 25 U/mL. A phorbol ester (for example, phorbol-12-myristate-13-acetate; PMA) may be added.

In the production method according to the present invention, the step of culturing a megakaryocyte may be carried out under at least one of a serum-free and a feeder cell-free condition. This is preferably a method carried out by culturing the megakaryocyte produced according to the method of the present invention in a medium containing TPO. If the platelet production step can be carried out serum-free and feeder cell-free, an immunogenicity problem is less likely to occur when the obtained platelet is used clinically. In addition, if a platelet can be produced without using a feeder cell, it is not necessary to adhere the feeder cell and thus suspension culture can be carried out in a flask or the like, and thus, the production cost can be minimized and such production is suitable for mass production. When no feeder cell is used, a conditioned medium may be used. The conditioned medium is not particularly limited and can be prepared by those skilled in the art according to a known method or the like, and for example, can be obtained by appropriately culturing a feeder cell and removing the feeder cell from the resulting culture by using a filter.

A ROCK inhibitor and/or an actomyosin complex function inhibitor may be added to the platelet production medium. Examples of the ROCK inhibitor and the actomyosin complex function inhibitor that can be used include the same as those used in the method for producing a multinucleated megakaryocyte as described above. Examples of the ROCK inhibitor include Y27632. Examples of the actomyosin complex function inhibitor include blebbistatin, which is a myosin heavy chain II ATPase inhibitor. A ROCK inhibitor may be added alone, a ROCK inhibitor and an actomyosin complex function inhibitor may be added alone, or these may be added in combination.

A ROCK inhibitor and/or an actomyosin complex function inhibitor is preferably added in an amount of 0.1 μM to 30 μM, and may be added in an amount of, for example, 0.5 μM to 25 μM, or 5 μM to 20 μM. The culture period after the addition of a ROCK inhibitor and/or an actomyosin complex function inhibitor can be 1 to 15 days, and may be 3 days, 5 days, 7 days, or the like. The proportion of a CD42b positive platelet can be further increased by adding a ROCK inhibitor and/or an actomyosin complex function inhibitor.

For the conditions for the culture composition and the like in the method for producing a platelet in the present step (a), US 2012/0238023 A1 (WO 2011/034073), US 2014/0127815 A1 (WO 2012/157586), and US 2016/0002599 A1 (WO 2014/123242), which disclose non-limiting examples of a method for producing a megakaryocyte and a method for producing a platelet can be referred to, and the disclosures of these Patent Applications are hereby incorporated by reference in their entirety.

The culture period specified in step (a) is at least 6 days. The term “at least 6 days” means about 144 hours or more. Therefore, the culture period may be, for example, 6 days, 6.5 days (about 156 hours), 7 days (about 168 hours), 7.5 days (about 180 hours), 8 days (about 192 hours), 8.5 days (about 204 hours), or 9 days (about 216 hours). In a certain embodiment, the culture period is at least 6 days and less than 8 days (about 192 hours). In another embodiment, the culture period is at least 6 days and less than 7 days (about 168 hours). During the culture period, it is desirable to carry out subculturing as appropriate.

During the culture period, a turbulent flow is generated in a medium containing a megakaryocyte. This is because by culturing a megakaryocyte in the presence of a turbulent flow, the megakaryocyte is “educated” to improve the production of a platelet from the megakaryocyte in both quality and quantity. A turbulent flow may be generated continuously or intermittently from the start to the end of the culture period. In a certain embodiment, the culture is carried out for at least 6 days (about 144 hours) under a condition in which a turbulent flow is continuously present. The culture period may be, for example, 6 days, 6.5 days (about 156 hours), 7 days (about 168 hours), 7.5 days (about 180 hours), 8 days (about 192 hours), 8.5 days (about 204 hours), or 9 days (about 216 hours). In particular, it is preferable to carry out the culture for 6 days under a condition in which a turbulent flow is continuously present. In addition, a turbulent flow is preferably generated such that the turbulent energy has a value of about 0.00016 m²/s² to about 0.02 m²/s². In addition, a turbulent flow is more preferably generated such that the shear stress has a value of about 0.1 Pa to about 6.0 Pa. The turbulent energy can also have a constant value from the start to the end of the culture period, but may be changed. Therefore, in a certain embodiment, the present invention relates to a method for improving a function of a megakaryocyte (that is, an ability thereof to produce a platelet), including culturing a megakaryocyte in the presence of a turbulent flow for at least 6 days. In another embodiment, the present invention relates to a method for producing a megakaryocyte having an improved function (that is, an improved ability to produce a platelet), including culturing a megakaryocyte in the presence of a turbulent flow for at least 6 days.

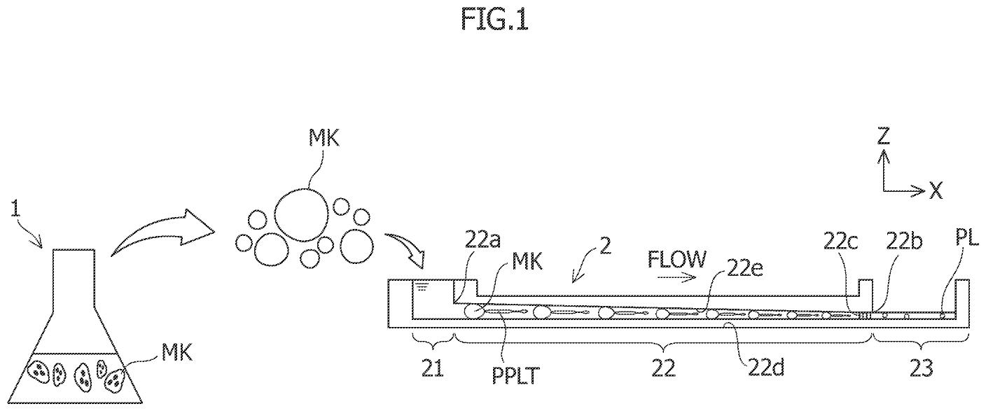

The method for generating a turbulent flow in a medium containing a megakaryocyte is not particularly limited. For example, as shown in FIG. 1, the method can be carried out using a flask 1. In this case, the method can be carried out by filling the flask 1 with a medium containing a megakaryocyte and shaking the flask for culturing. For example, the radius of gyration and the speed of rotation of a shaker that can generate the above preferable turbulent energy can be determined by a preliminary experiment, and the flask can be shaken according to the obtained conditions.

One example of the method for generating a turbulent flow in a medium containing a megakaryocyte may be a method involving using a culture vessel that can carry out unsteady stirring. More specifically, a culture vessel having an unsteadily operable blade can be used. The blade that is unsteadily operable is preferably a blade that can reciprocate up and down, reciprocate left and right, and/or reciprocate rotationally. Specific examples of the culture vessel that can be used include a VerMES reactor manufactured by Satake Machinery Co., Ltd. The VerMES reactor is described in detail in Patent Document 1, Non-Patent Document 1, WO 2017/077964, and WO 2019/009364, and the conditions and means disclosed therein can be used.

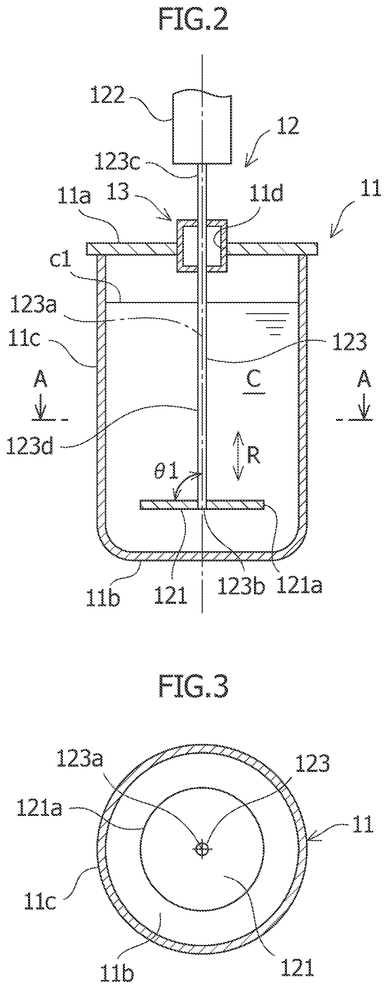

One example of the culture vessel that can carry out unsteady stirring will be briefly described with reference to FIGS. 2 and 3. The illustrated culture vessel and the operation of the culture vessel are examples. The culture vessel is not limited to one having a specific structure and carrying out a specific operation, as long as it is a culture vessel that can generate the predetermined turbulent energy. As shown in FIGS. 2 and 3, the culture vessel that can carry out unsteady stirring includes a container 11 for accommodating a medium C including a megakaryocyte, and a stirring mechanism 12 having one stirring blade 121 for stirring the medium C in the container 11. The stirring mechanism 12 is configured to reciprocate the stirring blade 121. In FIG. 2, the reciprocation direction of the stirring blade 121 is indicated by an arrow R. Furthermore, the stirring mechanism 12 controls the reciprocation of the stirring blade 121 in such a way as to generate the desired turbulent energy in the medium C. In the stirring mechanism 12, the stroke of the reciprocation of the stirring blade 121, the speed of the reciprocation (for example, the average speed of the reciprocation), the frequency of the reciprocation, and the like may be controlled. In particular, the reciprocation of the stirring blade 121 is preferably controlled in an unsteady pattern. The desired turbulent energy can be calculated by a known simulation technique.

Furthermore, the culture vessel is preferably configured as follows. The container 11 of the culture vessel is a hollow body, and in FIGS. 2 and 3, as one example, the container 11 is formed in a substantially cylindrical shape. In the present invention, the container may be formed in a shape other than a substantially cylindrical shape as long as the container is a hollow body. The container 11 has a top wall portion (or top portion) 11a and a bottom wall portion (or bottom portion) 11b that are opposed to each other substantially in a vertical direction and a peripheral wall portion (peripheral portion) 11c extending between outer peripheral edge portions of the top wall portion 11a and the bottom wall portion 11b. Furthermore, the container 11 is preferably formed in an elongated shape extending substantially in a vertical direction.

In FIG. 2, the top wall portion 11a is configured as a lid of the container 11, which is separate from the peripheral wall portion 11c, and the medium C can be fed to the inside of the container 11 in a state in which the top wall portion 11a is removed. In the present invention, a feed port for feeding the medium may be bored in the container, and in this case, the top wall portion may be formed integrally with the peripheral wall portion in the container. Furthermore, in the present invention, the container may be formed in such a way as to open upward according to the production condition of a platelet, and in this case, an opening may be formed in the top wall portion, or the container may have no top wall portion. The volume of the container 11 can be any value as long as a platelet can be produced, and for example, from the viewpoint of increasing the amount of platelets produced, the volume of the container 11 is preferably about 300 mL or more, about 1 L or more, about 50 L or more, about 200 L or more, about 500 L or more, about 1000 L or more, or about 2000 L or more.

As shown in FIG. 2, the stirring blade 121 of the stirring mechanism 12 of the culture vessel is disposed along an intersection plane that intersects with the reciprocation direction thereof at a predetermined intersection angle θ1. The intersection angle θ1 is about 90°. In other words, the stirring blade 121 is disposed along an intersection plane substantially orthogonal to the reciprocation direction thereof. The stirring blade 21 is formed in a substantially flat plate shape. The outer peripheral edge 121a of the stirring blade 121 is formed in a substantially circular shape when viewed from a direction orthogonal to the intersection plane. As shown in FIGS. 2 and 3, the stirring blade 121 is disposed at intervals from the top wall portion 11a, the bottom wall portion 11b, and the peripheral wall portion 11c of the container 11. Such a stirring blade 121 is also sometimes referred to as a “stirring impeller.” In addition, another shape of the stirring blade 121 and the distance between the peripheral wall portion 11c of the container 11 and the outer peripheral edge 121a of the stirring blade 121 may be determined according to the desired turbulent energy.

However, for the stirring blade of the present invention, the intersection angle of the stirring blade may be an intersection angle other than about 90° according to the desired turbulent energy. The intersection angle may be within the range of about 0° to about 180°. In addition, the stirring blade may be formed in a shape other than the substantially flat plate shape according to the desired turbulent energy, and for example, the stirring blade may be formed in a substantially hemispherical shell shape, a substantially bowl shape, a substantially curved plate shape, or a substantially corrugated plate shape. Furthermore, the outer peripheral edge of the stirring blade may be formed in a shape other than a substantially circular shape when viewed from a direction orthogonal to the intersection plane, according to the desired turbulent energy, and for example, the outer peripheral edge of the stirring blade may be formed in a substantially semicircular shape, a substantially elliptical shape, a substantially semi-elliptical shape, a substantially fan shape, a substantially polygonal shape such as a substantially quadrangular shape, a substantially star-shaped polygonal shape, or the like when viewed from a direction orthogonal to the intersection plane. The stirring blade may also have at least one hole penetrating in the reciprocation direction thereof, and the shape, number, and position of the hole may be determined according to the desired turbulent energy.

Furthermore, as shown in FIG. 2, the stirring mechanism 12 has a drive source 122 for reciprocating the stirring blade 121, and a connecting member 123 connecting the stirring blade 121 and the drive source 122. The drive source 122 is configured to reciprocate the stirring blade 121 by reciprocating the connecting member 122. In addition to the reciprocation, the drive source 122 may be configured to revolve the stirring blade 121 and the connecting member 123 around an axis line 123a of the connecting member 123. In this case, in the stirring mechanism 12, in addition to controlling the reciprocation of the stirring blade 121, the revolution speed, revolution direction, and the like of the stirring blade 121 may be controlled, and in particular, the reciprocation and revolution of the stirring blade 121 are preferably controlled in an unsteady pattern.

In addition, the connecting member 123 is formed in a substantially shaft shape extending along the axis line 123a thereof. A tip portion 23b in the longitudinal direction of the connecting member 123 is attached to the stirring blade 121, and a base end portion 122c in the longitudinal direction of the connecting member 123 is held by the drive source 22 in such a way as to be able to reciprocate. As shown in FIG. 2, a tip portion 123b of the connecting member 123 is attached at a position substantially coincident with the center of gravity of the stirring blade 121. The tip portion of the connecting member may be attached at a position deviating from the center of gravity of the stirring blade according to the desired turbulent energy.

The stirring mechanism 12 is attached to the top wall portion 11a of the container 11. For a specific attachment structure of the stirring mechanism 12, an insertion hole 11d penetrating in the reciprocation direction is formed in the top wall portion 11a of the container 11, and the stirring mechanism 12 is attached to the top wall portion 11a of the container 11 in a state in which the stirring blade 121 is accommodated inside the container 11 while inserting the connecting member 123 into the insertion hole 11d. In the present invention, the stirring mechanism may be attached to the bottom wall portion or the peripheral wall portion of the container by the above specific attachment structure of the stirrer instead of the top wall portion of the container.

When an attempt is made to improve the sealability of the container 11, the culture vessel may have a seal member 13 configured to close the gap between the peripheral edge of the insertion hole 11d of the container 1 and the connecting member 123 of the stirring mechanism 12 while allowing the reciprocation of the connecting member 123. For example, the seal member 13 may have a flexible structure that can follow the reciprocation of the connecting member 123. Furthermore, the flexible structure may be a film structure made of a flexible material such as rubber, or the flexible structure may be a bellows structure made of a metal, Teflon (registered trademark), or the like. In the present invention, the seal member may be configured to slidably hold the connecting member in the reciprocation direction.

In such a culture vessel, the stirring blade 121 of the stirring mechanism 12 reciprocates within a predetermined movable range in the container 11. The movable range is set in the container 11 or in the medium C so that the desired turbulent energy can be obtained. In particular, the length of the movable range in the reciprocation direction, that is, the maximum stroke of the reciprocation of the stirring blade 21, and the center position of the movable range in the reciprocation direction may be determined according to the length of the container 11 in the reciprocation direction, the distance from the bottom wall portion 11b of the container 11 to a liquid level c1 of the medium C, the volume of the container 11, and the desired turbulent energy.

The above culture vessel is one example of an apparatus for practicing the method of the present invention, and step (a) of the present invention is not particularly limited as long as the predetermined turbulent energy is applied to a platelet production medium containing a megakaryocyte.

The megakaryocyte obtained after the completion of step (a) is a population of cells having a non-uniform cell diameter and having a certain distribution in cell diameter. In general, the population is a cell population in which the distribution curve of a cell diameter has a single peak and shows an approximately lognormal distribution curve. In such a megakaryocyte cell population, the maximum diameter of the megakaryocytes is the maximum value obtained from measured values of the diameter of the megakaryocyte population included in the medium that has undergone step (a). Similarly, the minimum diameter of the megakaryocytes is the minimum value obtained from measured values of the diameter of the megakaryocyte cell population included in the medium that has undergone step (a). The shape of the cell diameter distribution curve in the megakaryocyte cell population, as well as the maximum diameter and the minimum diameter of the cells, are approximately the same in the megakaryocyte cell population cultured under the same conditions. The diameter of the megakaryocyte is, for example, about 5 to about 160 μm.

After the completion of step (a) and before step (b), for example, a step of removing a contaminant from the medium of step (a) by using a filter or the like or a step of exchanging the medium can also be carried out. Alternatively, the platelet production medium that has undergone step (a) can also be subjected to step (b). Within approximately 2 hours, preferably within 1 hour, after the completion of step (a), step (b) can be carried out.

Next, step (b) will be described. Step (b) is a step of injecting the megakaryocyte that has undergone the step of culturing into a predetermined platelet production device 2 and exposing the megakaryocyte to a laminar flow. This can mainly apply shear stress to the megakaryocyte and promote the production of a platelet from the megakaryocyte.

Here, a platelet production device that can carry out step (b) will be described with reference to FIG. 1. FIG. 1 is a cross-sectional view schematically showing a platelet production device 2 according to a first aspect of the present invention. As shown in FIG. 1, the platelet production device 2 includes an injection port 21 for a megakaryocyte cell population, a channel 22, and a platelet collection section 23. The channel 22 is configured such that one end 22a thereof communicates with the injection port 21 and the other end 22b communicates with the collection section 23. In FIG. 1, X represents the flow direction of the channel 22 of the platelet production device 2, and Z represents the height direction of the channel 22. Flow represents the direction of a flow that is applied to the megakaryocyte.

The injection port 21 of the platelet production device 2 is disposed corresponding to an upstream end portion 22a of the channel 22. The injection port 21 is an open portion that opens toward the outside of the device, and is configured such that the medium containing the megakaryocyte can be applied to the platelet production device 2 from the injection port 21. The shape and size of the injection port 21 are not particularly limited, and the injection port 21 can have a shape suitable for feeding the medium containing the megakaryocyte into the device 2 by using liquid feeding means, for example, a microtube or a pump.

The collection section 23 of the platelet production device 2 is disposed adjacent to a downstream end portion 22a of the channel 22. The collection section 23 can also be configured as an open portion that opens toward the outside of the device. The collection section 23 includes a space that can store the medium flowing in from the channel 22 and a platelet PL. In addition, the collection section 23 can have a shape suitable for collecting the stored platelet from the device 2 by using collection means, for example, a pipette, a microtube, or a pump.

The channel 22 is a space that extends from the injection port 21 toward the collection section 23 and that is configured to allow a fluid to pass therethrough. The channel 22 may have a height defined by the distance between a bottom surface 22d and a top surface 22e, and it may have a shape in which the shape of a cross section perpendicular to the flow direction is substantially rectangular. The channel 22 is configured such that the height of an end of the channel 22a on the injection port side is greater than the maximum diameter of a megakaryocyte. The height of an end of the channel 22b on the collection section side is at least less than the minimum diameter of a megakaryocyte and greater than the maximum diameter of a platelet. Then, the height of the channel decreases from the injection port 21 toward the collection section 23. The height of the end of the channel 22a on the injection port side is not limited to a specific value. In a certain embodiment, the height of the channel can be determined based on a probability of top about 0.05% of a lognormal distribution curve for the particle size of the megakaryocyte. The height of the channel is configured to decrease from the injection port 21 toward the collection section 23. Therefore, herein, the height of the end of channel 22a on the injection port side is also sometimes referred to as the maximum channel height (h_{_max}), and the height of the end of channel 22b on the collection section side is also sometimes referred to as the minimum channel height (h_{_min}). The height of the channel preferably decreases monotonically from the injection port 21 toward the collection section 23, and may decrease linearly or exponentially. However, the height of the channel is preferably constant in the width direction of the channel. That is, when the length from the end portion 22a on the injection port side to the end portion 22b on the collection section side is a length l_{c} of the channel, the height h(x) of the channel at a predetermined distance x from the end portion 22a on the injection port side along the length direction of the channel is a constant value in the width direction. The width of the channel may be constant or change from the end portion 22a on the injection port side to the end portion 22b on the collection section side. The changing embodiment will be described in detail later as the platelet production device according to a second aspect.

Optionally, the channel 22 preferably includes a plurality of capture pillars 22c rising from the bottom surface 22d in the vicinity of the end portion 22b on the collection section side. When the megakaryocyte produces a platelet, the megakaryocyte forms stretched platelet precursors PPLT (proplatelets), which may be cleaved by a shear force. By providing the capture pillars 22c at the above location, the stretched string-shaped platelet precursors are caught and captured, the platelet precursors are prevented from flowing out from the channel 22 to the collection section 23, and platelets can continue to be produced.

The vicinity of the end portion 22b on the collection section side can be particularly referred to as a portion in which the height of the channel is smaller than the size of the megakaryocyte. However, the capture pillars 22c may also be provided at a location other than the above location.

The size of the capture pillars 22c and the interval therebetween are not particularly limited as long as they can capture the platelet precursors, and can be appropriately determined in consideration of the flow rate of a liquid flowing through the channel 22 and the like. If the interval between the capture pillars 22c is too large, the platelet precursors may slip through the interval without being caught, and if the interval is narrowed and the capture pillars 22c are densely formed, the resistance of a fluid flowing through the channel 22 may increase. The interval between the capture pillars 22c can be appropriately determined in consideration of the pressure of a liquid flowing through the channel 22 and the like. When forming the capture pillars 22c, the flow rates of a liquid flowing through the portions in which the capture pillars 22c are disposed may be designed to be the same so that the shear forces applied to the captured platelet precursors are the same.

By using the platelet production device 2 including the capture pillars 22c, a platelet can be produced more efficiently.

As shown in FIG. 1, the platelet production device 2 may also have a channel extending in one direction from the injection port 21. Alternatively, the platelet production device 2 may also include a plurality of channels radially extending around the injection port 21 in the outer peripheral direction of the injection port 21. Furthermore, the platelet production device 2 may also include a channel extending 360 degrees around the injection port 21 in the circular shape in the outer peripheral direction of the injection port 21. Specific examples of the channel extending 360 degrees in the outer peripheral direction of the injection port 21 include the platelet production device disclosed in Patent Document 2 by the present inventors. The platelet production device disclosed in Patent Document 2 also has a predetermined channel shape and can apply shear stress to a megakaryocyte for a predetermined time in a state in which the megakaryocyte is captured, and the platelet production device disclosed in Patent Document 2 can be used in step (b) of the present invention. The material of the platelet production device is not particularly limited, and examples thereof include a synthetic polymer such as polyethylene, polypropylene, polystyrene, an acrylic resin, an epoxy resin, a silicone resin, polycarbonate, or polyvinyl chloride, an inorganic material such as a glass (borosilicate glass or the like), silicon, alumina, or titania, a metal such as stainless steel, titanium, or aluminum, and a photoresist (photosensitive resin).

A method for carrying out step (b) using the above platelet production device will be described. Step (b) can be mainly composed of the following substeps:
(i) a loading step of injecting the medium containing the megakaryocyte that has undergone the culture of step (a) into the platelet production device 2;
(ii) a producing step of injecting a medium containing no megakaryocyte or a fluid that can form a laminar flow into the platelet production device 2; and
(iii) a flushing step

The flushing step is an optional step and need not be carried out.

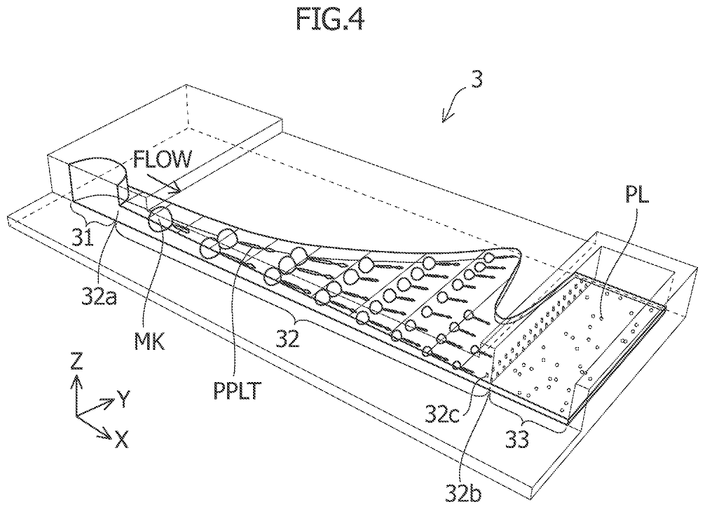

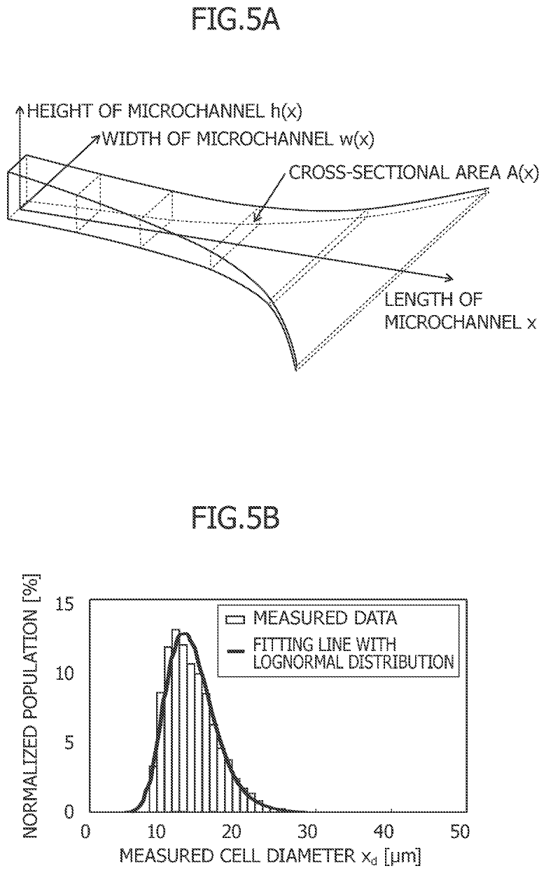

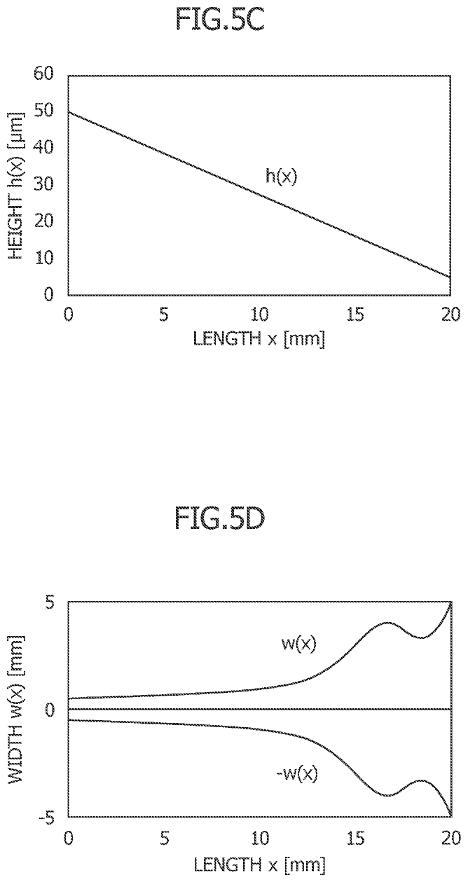

In substep (i), the medium containing the megakaryocyte is injected into the platelet production device 2. Thereby, the megakaryocyte is captured in the channel in the vicinity of a location having a height of the channel suitable for the diameter of the megakaryocyte. The medium injection can be carried out under pressure such that the medium has a predetermined flow rate in the channel of the platelet production device. In substep (i), a preferable flow rate is about 0.1 to 5 mm/s. The pressure at that time varies greatly depending on the shape and specifications of the platelet production device, and thus, the injection is appropriately carried out at a pressure that can achieve the preferable flow rate in the device used. For example, when the device described with reference to FIGS. 4 and 5A to 5D, described later, is used, the pressure can be about 1 to 200 KPa, but the pressure is not limited to a specific value. The substep (i) is preferably carried out in a state in which the device is kept at about 37° C. The time required for the loading step can be appropriately determined by those skilled in the art depending on the total amount of the medium containing the megakaryocyte to be injected into the platelet production device 2 and the like. As one example, the time can be about 10 to 20 minutes, but it is not limited to a specific time.

In the substep (ii), a medium containing no megakaryocyte or other fluid is injected into the platelet production device 2. The fluid is not particularly limited as long as it is a fluid that can form a laminar flow in the channel and does not adversely affect the function of the megakaryocyte. Examples of the fluid include, but are not limited to, saline or phosphate buffered saline. Examples of the medium containing no megakaryocyte include, but are not limited to, a medium obtained by removing the megakaryocyte and the platelet from the supernatant of the medium used in step (a) by using a filter or the like. The medium or the fluid can be injected under pressure such that the medium or the fluid has a predetermined flow rate in the channel of the platelet production device. In substep (ii), a preferable flow rate is about 0.1 to 5 mm/s as in substep (i). Therefore, the pressure at that time can be determined in the same manner as in substep (i), and for example, when the device described with reference to FIGS. 4 and 5A to 5D described later is used, the pressure may be in the same pressure range as in substep (i). In a state in which the device is kept at about 37° C., substep (ii) can also be carried out, for example, for 1 to 10 hours, and can also be carried out for 4 to 6 hours, but the time required therefor is not limited to a specific time range. During the carrying out the production step of substep (ii), the megakaryocyte is exposed to a medium or the fluid while still captured in the channel. Thereby, shear stress is applied to the megakaryocyte. Thereby, the megakaryocyte is stretched to form a platelet precursor, and furthermore, a platelet is produced from the platelet precursor. The produced platelet may also flow through the channel and reach the collection section. In addition, a part thereof flows through the channel in the state of a platelet precursor. The capture pillars 22c, which may optionally be provided in the latter part of the channel, capture a platelet precursor and promote platelet production. A plurality of megakaryocytes may include one not exhibiting the behavior described in this paragraph.

In substep (iii) which is an optional step, the same fluid as in substep (ii) is injected into the platelet production device 2 at the same pressure as, or at a higher pressure than, in substep (ii). At this time, a preferable flow rate of the fluid in the channel is about 5 to 50 mm/s. For example, when the device described with reference to FIGS. 4 and 5A to 5D, described later is used, the pressure for achieving this flow rate can be about 50 to 200 KPa, but the pressure is not limited to a specific pressure value. In a state in which the device is kept at about 37° C., substep (iii) can also be carried out, for example, for 10 to 20 minutes, but the time required therefor is not limited to a specific time range. By carrying out substep (ii), a platelet can usually be produced and collected, but this operation can also be additionally carried out. In FIG. 4, Flow represents the flow direction of the fluid in the vicinity of the injection port.

Next, examples of a second aspect of the platelet production device include a device having the above configuration and further having a configuration in which the width of the channel changes. Another aspect of the platelet production device will be described with reference to FIG. 4. A device 3 shown in FIG. 4 has a configuration including an injection port 31 for a megakaryocyte, a platelet collection section 33, and a channel 32 extending from the injection port to the collection section and characteristics of the height of the channel in common with the platelet production device 2 shown in FIG. 1. In addition, the device 3 also has capture pillars 33c, which may be optionally provided, in common with the platelet production device 2 shown in FIG. 1. In FIG. 4, X represents the flow direction of the channel 32 of the platelet production device 3, Y represents the width direction of the channel 32, and Z represents the height direction of the channel 32.

In the present aspect, the width of the channel 32 changes from the injection port 31 toward the collection section 33, and the change correlates with the frequency distribution of the diameter of a megakaryocyte population injected. More specifically, when the diameter of the megakaryocyte is the diameter x_{d}, the distance from the end portion on the injection port side is x, the height of the channel at the distance x is h(x), and the width of the channel at the distance x is w(x), w(x) is determined according to the diameter distribution of a megakaryocyte having a diameter of h(x), and the channel is configured such that w(x) increases as the frequency of the megakaryocyte having a diameter x_{d} of h(x) increases. By configuring the device 3 as described above, the device 3 has the same functions as described with reference to FIG. 1, and furthermore, even after the megakaryocyte cell population is captured in the channel 32, constant fluid conditions, for example, a constant flow rate, can be held.

The design of the width of the channel will be described in more detail with reference to FIGS. 5A to 5D. The diameter x_{d} of a megakaryocyte is represented by the following Expression (1) because the probability density function P(x_{d}) is considered to follow a lognormal distribution.

$$\begin{matrix}Expression 1 & \\ P ( {x}_{d} ) = \frac{1}{\sqrt{2 {πσ}_{d}^{2} {x}_{d}}} exp ( - \frac{{( ln {x}_{d} - {μ}_{d} )}^{2}}{2 {σ}_{d}^{2}} ) & ( 1 )\end{matrix}$$

In the Expression (1), μ_{d} and σ_{d} as represent the average and the standard deviation, respectively, in the normal distribution as the function ln(x_{d}). By estimating this distribution, the probability of a megakaryocyte having a predetermined diameter x_{d} can be predicted.

In the present aspect, the channel width is designed to reflect the cell size distribution. Here, a case in which an attempt is made to capture a megakaryocyte group having a diameter of x_{d} by using a channel having a maximum width of w_{c} and a maximum length of l_{c} will be studied. In this case, x_{d} takes a value in the range of x_{d_min} or more and x_{d_max} or less. Here, the cross-sectional area of the channel at the distance x from the end portion on the injection port side, 33a, of the channel 33 is defined as A(x), the height of the channel thereat is defined as h(x), and the width of the channel thereat is defined as 2w(x). FIG. 5A is a diagram showing descriptions of the defined variables. In FIG. 5A, the channel has a shape symmetrical with respect to axis x. In addition, although not shown, the starting points of the arrow x, w(x), and h(x) correspond to the end portion on the injection port side, 33a, of the channel 33.

Considering that a megakaryocyte having a diameter x_{d} is captured at a position at the distance x from the end portion on the injection port side end, 33a, at a location having a specific channel cross-sectional area, the height h(X) of the channel is represented by the following Expression (2).

Expression 2

h(x)=x_{d} (2)

Considering a case in which the height h(x) of the channel decreases linearly with the length x of the channel, x_{d} is obtained by the following Expression (3).

Expression 3

x=x_{d_max}−Sₗₒₚₑx (3)

In the Expression (3) Sₗₒₚₑ is the slope of the channel in the height direction and is represented by the following Expression (4).

$$\begin{matrix}Expression 4 & \\ {S}_{lope} = \frac{{x}_{d \_ max} - {x}_{d \_ min}}{{l}_{c}} & ( 4 )\end{matrix}$$

In this configuration, the megakaryocyte cell population injected into the device is sequentially captured depending on the diameter x_{d} thereof, from a large megakaryocyte x_{d_max} to a small megakaryocyte x_{d_min}. Then, the cross-sectional area A(x) at the distance x is reduced by the captured megakaryocytes having a diameter x_{d}. Here, when the reduced cross-sectional area is A_{dec}, the effective cross-sectional area Ad(x) that allows the passage of the megakaryocyte medium is represented by the following Expression (5).

$$\begin{matrix}Expression 5 & \\ \begin{matrix}{A}_{ef} ( x ) = A ( x ) - {A}_{dec} ( x ) \\ = 2 h ( x ) w ( x ) - {A}_{dec} ( x )\end{matrix} & ( 5 )\end{matrix}$$

The effective cross-sectional area is changed by the decrease w_{dec}(x) in the channel width caused by the capture of the megakaryocyte, and thus, the effective channel width w_{ef}(x) is represented by the following Expression (6).

Expression 6

w_{ef}(x)=w(x)−w_{dec}(x) (6)

Here, considering the probability density function P(x_{d}), the decrease w_{dec}(x) in the channel width is represented by the following Expression (7).

$$\begin{matrix}Expression 7 & \\ {w}_{dec} ( x ) = {x}_{d} N \frac{P ( {x}_{d} )}{2} & ( 7 )\end{matrix}$$

In the Expression (7) N represents the total number of megakaryocytes included in the megakaryocyte cell population.

If the effective cross-sectional area A_{ef}(x) is designed to be constant in order to keep the flow rate in the channel constant, the following Expression (8) is obtained.

Expression 8

A_{ef}(x)=2h(x)w_{ef}(x)=Const. (8)

Then, based on the maximum length l_{c} of the channel and Expressions (5) to (8), the following Expression (9) is obtained.

$$\begin{matrix}Expression 9 & \\ \begin{matrix}{A}_{ef} ( x ) = 2 ( w ( x ) - {x}_{d} N \frac{P ( {x}_{d} )}{2} ) h ( x ) \\ = 2 ( w ( {l}_{c} ) - {x}_{d \_ min} N \frac{P ( {x}_{d \_ min} )}{2} ) h ( {l}_{c} )\end{matrix} & ( 9 )\end{matrix}$$

Therefore, as a function of the channel length, the channel width can be derived as shown in the following Expression (10).

$$\begin{matrix}Expression 10 & \\ w ( x ) = ( w ( {l}_{c} ) - {x}_{d \_ min} N \frac{P ( {x}_{d \_ min} )}{2} ) \frac{h ( {l}_{c} )}{h ( x )} + {x}_{d} \frac{P ( {x}_{d} )}{2} & ( 10 )\end{matrix}$$

In the Expression (10), h(l_{c}) and w(l_{c}) are determined by the sizes x_{d_min} and w_{c}, respectively, of the channel. Finally, the design of the channel can be represented by the following Expressions (11) and (12) based on Expressions (2) to (4) and Expression (10).

$$\begin{matrix}Expression 11 & \\ h ( x ) = {x}_{d \_ min} - {S}_{lope} x & ( 11 )\end{matrix}$$

$$\begin{matrix}Expression 12 & \\ w ( x ) = ( {w}_{c} - {x}_{d \_ min} N \frac{P ( {x}_{d \_ min} )}{2} ) \frac{{x}_{d \_ min}}{{x}_{d \_ min} - {S}_{lope} x} + ( {x}_{d \_ min} - {S}_{lope} x ) N \frac{P ( {x}_{d \_ min} - {S}_{lope} x )}{2} & ( 12 )\end{matrix}$$

FIG. 5B is one example of the diameter distribution after culturing, in step (a), of a megakaryocyte group induced to differentiate from a human pluripotent stem cell, and is a measurement result of 10314 cells cultured in five dishes. This result was fitted to the lognormal distribution of Expression (1) by the least squares method. Then, μ_{d} was 2.99 μm and σ_{d} was 0.38 μm. Next, x_{d_min} was set to 5 μm based on the fact that the diameter of a normal platelet is less than 3 μm. In addition, x_{d_max} was set to 50 μm based on a probability of it being in the top about 0.1% of the normal distribution. Next, for a reason for producing the device, w_{c} was set to 10 mm and l_{c} was set to 20 mm. From these values and Expressions (11) and (12), the three-dimensional shape of the channel can be obtained. A graph of h(x) designed based on the diameter distribution of the megakaryocyte group shown in FIG. 5B is shown in FIG. 5C, and a graph of w(x) is shown in FIG. 5D.

The platelet production device designed as described above can be produced using a 3D printer or a photoresist forming technique.

As described above, the platelet production device according to the second aspect of the present invention can be designed fit a specific megakaryocyte group cultured under a specific condition. Therefore, the production method of the present invention may optionally include, before step (a), a step of designing and producing a platelet production device to fit a desired megakaryocyte group.

The method for carrying out step (b) using the platelet production device according to the second aspect of the present invention may be the same as that described in the first aspect.

The platelet production device according to the second aspect of the present invention has the above characteristics of the channel width, and thus, even after a megakaryocyte is captured in the channel, a constant fluid condition (flow rate) of the fluid can be held in the channel. Therefore, particularly by using a platelet production device, a medium or the like can be kept under a constant fluid condition in the producing step, which is a substep of step (b), thereby reducing variation in the number of platelets produced.

After step (b), a platelet collection step can be carried out. In the platelet collection step, the platelet-containing medium stored in the collection section is taken by means such as a pipette or a pump, and the platelet is collected from the medium by a usual method such as FACS. The “platelet” is one of the cellular components in blood and is characterized by being CD41a positive and CD42b positive. The platelet plays an important role in thrombus formation and hemostasis, and it is also involved in tissue regeneration after injury and the pathophysiology of inflammation. When a platelet is activated by bleeding or the like, a receptor for a cell adhesion factor such as Integrin αIIBβ3 (glycoprotein IIb/IIIa; a complex of CD41a and CD61) is expressed on the membrane thereof. As a result, platelets aggregate with each other, and fibrin is coagulated by various blood coagulation factors released from the platelets, thereby forming a thrombus to promote hemostasis.

The function of a platelet can be measured and evaluated by a known method. For example, the amount of an activated platelet can be measured using an antibody to PAC-1 that specifically binds to Integrin αIIBβ3 on the membrane of the activated platelet. In addition, similarly, CD62P (P-selectin), which is a platelet activation marker, may be detected using an antibody to measure the amount of an activated platelet. For example, the measurement of the amount thereof can be carried out by carrying out gating using an antibody against the activation-independent platelet marker CD61 or CD41 using flow cytometry, and then detecting the binding of an anti-PAC-1 antibody or an anti-CD62P antibody. These steps may also be carried out in the presence of adenosine diphosphate (ADP).

In addition, the function of a platelet can also be evaluated by observing whether or not the platelet binds to fibrinogen in the presence of ADP. The binding of a platelet to fibrinogen causes the activation of an integrin required early in thrombus formation. Furthermore, the function of a platelet can also be evaluated by a method involving visualizing and observing the ability to form a thrombus in vivo, as disclosed in WO 2011/034073.

The platelet obtained by the production method of the present invention can be administered to a patient as a preparation. For the administration, the platelet obtained by the method of the present invention may be preserved in, for example, human plasma, an infusion solution, a citric acid containing saline solution, a solution containing glucose-acetated Ringer's solution as the main agent, or PAS (platelet additive solution) (Gulliksson, H., et al., Transfusion, 32:435-440, (1992)) and formulated. The preservation period is about 3 to 7 days, for example, about 4 days. As a preservation condition, it is desirable to preserve the platelet by shaking and stirring at room temperature (about 20 to 24 degrees).

The present invention has been completed based on the following findings: at least 6 days of culture, not 5 days, is required in education for turbulent flow-dependent maturation of a megakaryocyte; and if even pre-education for maturation into a megakaryocyte can be carried out, a platelet having functionality can be efficiently produced in a subsequent shear stress-dependent platelet production process. According to the production method of the present invention, a platelet sufficiently having properties that allow administration as a blood product can be efficiently produced.

### EXAMPLE

Hereinafter, the present invention will be described in more detail using an Example of the present invention. The following Example does not limit the present invention.

### 1. Production of Platelet Production Device

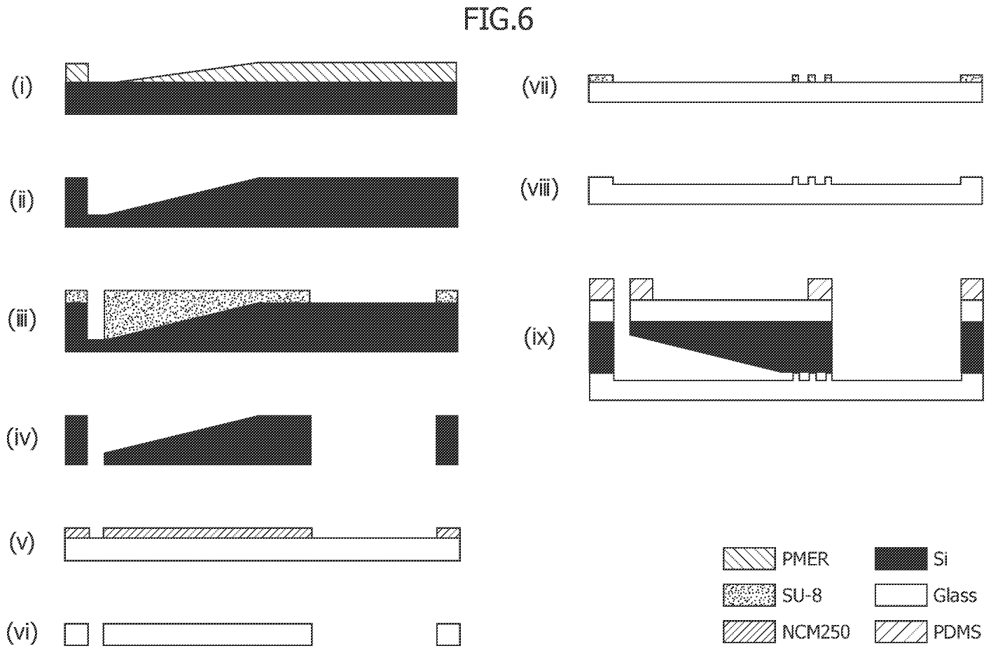

The platelet production device is outlined in FIG. 4, was designed according to FIGS. 5A to 5D, and is produced as follows. FIG. 6 is a diagram schematically showing the production of the device. A platelet production device composed of four layers: a cover layer, a 3D channel layer, a holder layer, and a polydimethylsiloxane (PDMS) layer, was produced. The production process is as follows. In FIG. 6, (i) to (iv) are cross-sectional views showing the production of the 3D channel layer, (v) and (vi) are cross-sectional views showing the production of the cover layer, and (vii) and (viii) are cross-sectional views showing the production of the holder layer; (ix) shows a platelet production device in which four layers are integrated and packaged. PMER, SU-8, NCM-250, Si, Glass, and PMDS represent the materials that make up each layer in the figure, and details thereof are shown below.
(i) As the 3D channel layer, a positive photoresist, PMER (TOKYO OHKA CO., LTD.), was patterned on the surface of a Si substrate using a grayscale lithography technique. In this process, a pattern designed on an 8-bit gray scale by laser scanning was directly formed by exposure by varying the laser intensity.
(ii) The Si substrate was etched using D-RIE-CSR (deep reactive ion etching with controlled selective ratio). The 3D surface of the photoresist was transferred to the Si substrate according to the selectivity.
(iii) Next, a negative photoresist, SU-8 3010 (Microchem Co. Ltd, Japan), was patterned on the Si substrate.

The SU-8 layer was used as an etching mask for the D-RIE process to produce an injection port and a collection section of the platelet production device.
(iv) The injection port and the collection section were produced using D-RIE, and then the residual photoresist was removed by a cleaning process.
(v) As the cover layer, a negative photoresist, NCM-250 (Nikko-Materials Co. Ltd, Japan), was patterned on borosilicate glass as an etching mask for sandblasting.
(vi) The borosilicate glass was etched by sandblasting. In this process, an injection port and a collection section were produced.
(vii) For the holder layer, SU-8 3010 was patterned on borosilicate glass.
(viii) The borosilicate glass was etched using RIE to obtain a structure having a height of 5 μm corresponding to x_{d_min} and a micropillar region.
(ix) Finally, three processed layers of the cover, 3D channel, and holder layers were bonded by an anodic bonding technique. The injection port and the PDMS portion of a collection chamber were bonded as a PDMS layer on the cover layer.

### 2. Production of Platelets

Platelets were produced by the production method of the present invention, and properties thereof were evaluated.

imMKCL (Megakaryocyte Cell Line) Gene ON Proliferation Culture

An imMKCL was cultured at 37° C. in a 5% CO₂ atmosphere in a medium obtained by supplementing an imMKCL differentiation medium (15% FBS, L-glutamine, insulin-transferrin-selenium, ascorbic acid, and 1-thioglycerol in IMDM medium) with 50 ng/mL SCF, 200 ng/ml TA-316, and 1 μg/ml Doxycycline.

imMKCL (Megakaryocyte Cell Line) Gene OFF Maturation Culture (Step a)

An imMKCL was cultured in a medium obtained by supplementing an imMKCL differentiation medium with 50 ng/mL SCF, 200 ng/mL TA-316, 15 mM KP-457, 0.75 mM SR-1, and 10 mM Y27632 for 5 days, 6 days, 7 days, or 8 days at 37° C. in a 5% CO₂ atmosphere, at a cell density of 1×10 e⁵/ml, at 100 rpm in 125 mL Corning_Erlenmeyer cell culture flasks using a culture device, Lab-Therm shaker.

### Platelet Production Using Platelet Production Device (Step b)

As the platelet production device, one produced by the method as described above and shown in FIG. 4 was used. This platelet production device 3 is composed of an injection port 31 for introducing a megakaryocyte and a medium, a channel 32 for capturing a megakaryocyte, a capture micropillar 32c for capturing a platelet precursor cell rarely torn from a megakaryocyte, and a collection section 33 for collecting a platelet produced. The medium containing a megakaryocyte on day 5, 6, 7, or 8 of the Gene OFF maturation culture was loaded into the injection port at 10 kPa for 15 minutes (substep (i) loading step). Thereby, 2500 megakaryocytes were introduced into the platelet production device. In an experiment for evaluation of the number produced, the medium containing megakaryocytes was introduced into the device, then the pressure was kept at 10 kPa, and only the medium for the production step including no megakaryocyte was caused to flow for 6 hours to apply shear stress to the megakaryocytes (substep (ii) production step). The medium for the production step was a medium obtained by removing the megakaryocytes and the platelets from the culture supernatant used in the Gene OFF maturation culture step by using a 0.22 μm filter. Therefore, when the megakaryocytes that had undergone the Gene OFF maturation culture for 5 days were used with the platelet production device, the culture medium for the production step in substep (ii) production step at that time was a medium obtained by removing the megakaryocytes and the platelets from the culture supernatant after 5 days of the Gene OFF maturation culture. Similarly, when the megakaryocytes that had undergone the Gene OFF maturation culture for 6, 7, or 8 days were used with the platelet production device, the culture medium for the production step at that time was a medium obtained by removing the megakaryocytes and the platelets from the culture supernatant after 6, 7, or 8 days, respectively, of the Gene OFF maturation culture, respectively. Next, the platelet mixed culture solution that had gathered in the collection section located downstream of the channel was collected, and the number of platelets was measured using FACS Verse. In a platelet function measurement experiment, the medium containing megakaryocytes was loaded, then the pressure was kept at 10 kPa, and the same medium for the production step as that used in the experiment for evaluation of the number produced was caused to flow for 1 hour to apply shear stress to the megakaryocytes. After that, the platelet mixed culture solution that had gathered in the collection section located downstream of the channel was collected, and the platelet hemostatic function was measured. In the present Example, substep (iii) flushing step was not carried out.

### Flow Cytometric Analysis

For flow cytometric analysis, FACS Verse was used. The antibodies used were anti-hCD41-APC (#303710), anti-hCD42b-PE (#303906), anti-hCD42a-PE (#558819), anti-hCD62PAPC (#304910), and FITC Annexin V (#556419) antibodies. For platelet activation reaction in PAC-1/p-selectin positive measurement, 40 mM TRAP-6 and 100 mM ADP were added. For Annexin V positive measurement, 20 mM ionomycin was used.

### Results

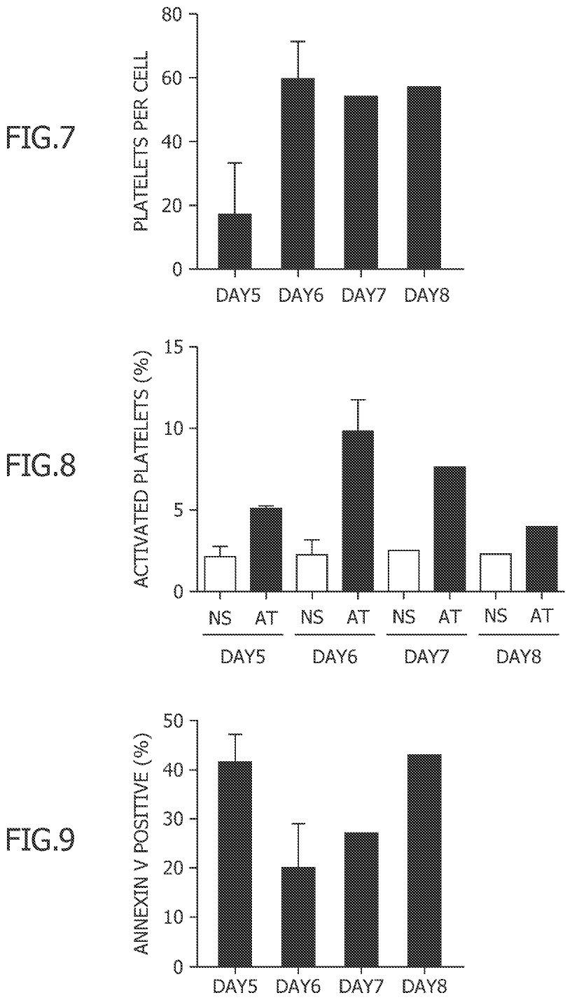

FIG. 7 shows the number of CD41a/CD42b positive platelets produced when megakaryocytes on days 5, 6, 7, and 8 of the Gene OFF maturation culture were introduced into the platelet production device to produce platelets. As a result of measuring the number of platelets, the number of platelets per imMKCL on day 5 of the Gene OFF maturation culture was about 16, whereas the number of platelets per imMKCL on day 6 of the culture was about 59, which was 3 or more times greater and indicated a dramatic improvement. The number of platelets per imMKCL on day 7 was about 54, and the number of platelets per imMKCL on day 8 was about 55. The number of platelets per imMKCL here referred to is the number produced by introduction into the platelet production device, and is the result of subtracting the number of platelets that had already been produced when the Gene OFF maturation culture was completed from the number of platelets finally produced.

FIG. 8 shows results obtained by introducing megakaryocytes on days 5, 6, 7, and 8 of the Gene OFF maturation culture into the platelet production device to produce platelets, collecting a platelet mixed culture solution, and measuring the platelet hemostatic function (PAC-1 positive). The imMKCL-derived platelets on day 5 of the Gene OFF maturation culture had a PAC1 positive rate of about 2.3% with no stimulation (NS), and had a PAC1 positive rate of about 5.1% at the time of addition of ADP/TRAP (AT), which is a platelet activation factor. In contrast, the imMKCL-derived platelets on day 6 of the Gene OFF maturation culture had a PAC1 positive rate of about 2.2% with no stimulation (NS) and a PAC1 positive rate of about 10% at the time of addition of ADP/TRAP (AT), indicating an improvement in the reaction of the platelet activation factor. In addition, the imMKCL-derived platelets on day 7 of the Gene OFF maturation culture had a PAC1 positive rate of about 2.5% with no stimulation (NS) and a PAC1 positive rate of about 7.6% at the time of addition of ADP/TRAP (AT), and the imMKCL-derived platelets on day 8 of the Gene OFF maturation culture had a PAC1 positive rate of about 2.3% with no stimulation (NS) and a PAC1 positive rate of about 4% at the time of addition of ADP/TRAP (AT).

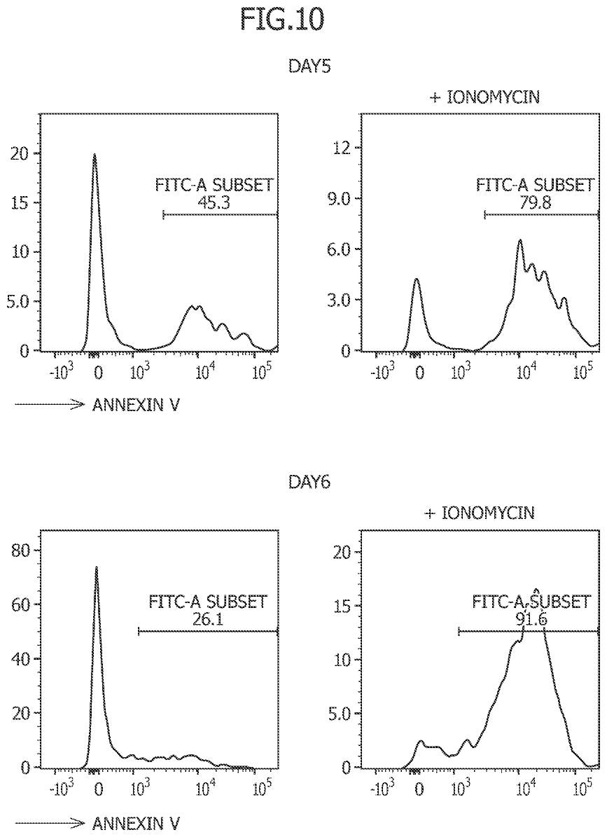

FIG. 9 shows results obtained by introducing megakaryocytes on days 5, 6, 7, and 8 of the Gene OFF maturation culture into the platelet production device to produce platelets, collecting a platelet mixed culture solution, and measuring Annexin V, which is a platelet aging marker. The imMKCL-derived platelets on day 5 of the Gene OFF maturation culture had an Annexin V positive rate of about 41%, whereas the imMKCL-derived platelets on day 6 of the Gene OFF maturation culture had an Annexin V positive rate of about 20%, indicating that these were Annexin V low positive platelets. The imMKCL-derived platelets on day 7 of the Gene OFF maturation culture had an Annexin V positive rate of about 26%, and the imMKCL-derived platelets on day 8 of the Gene OFF maturation culture had an Annexin V positive rate of about 43%. FIG. 10 is FACS diagrams showing Annexin V measurement results. The horizontal axis represents Annexin V, and an Annexin V positive gate was set based on the main population at the time of addition of ionomycin, which is a positive control.

As used herein and in the claims, a term in a singular form shall encompass that in a plural form and a term in a plural form shall encompass that in a singular form, unless specifically required in the context. Therefore, it should be understood that singular articles (for example in English, “a,” “an,” “the,” and the like) also encompass a concept thereof in a plural form unless otherwise noted. The term “about” generally refers to a range of numbers that those skilled in the art would consider to be equivalent to (that is, have the same function or result as) the value that follows. For example, the term “about” refers to ±10%, ±5%, or ±2% of the displayed value.

### INDUSTRIAL APPLICABILITY

The method for producing a platelet and the platelet production device according to the present invention are useful in the production of a blood product.

### REFERENCE SIGNS LIST

1 flask
11 container
12 stirring mechanism
2, 3 platelet production device
21, 31 injection port
22, 32 channel
23, 33 collection section

1. A method for producing a platelet, comprising the steps of:
(a) culturing a megakaryocyte for at least 6 days in a platelet production medium in which a turbulent flow is generated; and
(b) injecting the medium comprising the megakaryocyte that has undergone step (a) into a platelet production device to expose the megakaryocyte to a laminar flow, wherein the platelet production device comprises
an injection port for a megakaryocyte,
a platelet collection section, and
a channel extending from the injection port to the collection section, the channel is configured such that
a height of an end of the channel on the injection port side is greater than a maximum diameter of a megakaryocyte to be injected,
a height of an end of the channel on the collection section side is less than a minimum diameter of a megakaryocyte to be cell injected and greater than a maximum diameter of a platelet, and
the height of the channel decreases from the injection port toward the collection section,
and thereby the platelet production device is configured to make it possible to expose the megakaryocyte to the laminar flow in a state in which the megakaryocyte is captured in the channel and make it possible to release a platelet produced by the megakaryocyte from the channel into the collection section.

2. The method according to claim 1, wherein a width of the channel changes from the injection port toward the collection section, and the change correlates with a diameter distribution of the megakaryocyte to be injected.

3. The method according to claim 2, wherein when a distance of the channel from the end portion on the injection port side is x, a height of the channel at the distance x is h(x), a width of the channel at the distance x is w(x), and a diameter of the megakaryocyte is x_{d}, w(x) is determined according to a frequency of a megakaryocyte having a diameter of h(x), and the channel is configured such that w(x) increases as a frequency of a megakaryocyte having a diameter x_{d} of h(x) increases.

4. The method according to claim 1, wherein the platelet production device comprises a plurality of pillars rising from a bottom surface of the end portion in the channel on the collection section side.

5. The method according to claim 1, wherein the method comprises, before the step of culturing a megakaryocyte, a step of forcibly expressing an oncogene, a polycomb gene, and an apoptosis suppressor gene in a cell more undifferentiated than a megakaryocyte to obtain an immortalized megakaryocyte.

6. The method according to claim 1, wherein the method comprises a step of collecting a platelet from the collection section of the platelet production device.

7. The method according to claim 1, wherein the step of culturing a megakaryocyte for at least 6 days is carried out using a shaking flask or a culture vessel comprising an unsteadily operable blade.

8. A platelet production device comprising:
an injection port for a megakaryocyte;
a platelet collection section; and
a channel extending from the injection port to the collection section, wherein the channel is configured such that
a height of an end of the channel on the injection port side is greater than a maximum diameter of a megakaryocyte to be injected,
a height of an end of the channel on the collection section side is smaller than a minimum diameter of a megakaryocyte injected and greater than a maximum diameter of a platelet, and
the height of the channel decreases from the injection port toward the collection section, and
thereby, the platelet production device is configured to make it possible to expose the megakaryocyte to a laminar flow in a state in which the megakaryocyte is captured in the channel, and make it possible to release a platelet produced by the megakaryocyte from the channel into the collection section.

9. The device according to claim 8, wherein when a distance of the channel from the end portion on the injection port side is x, a height of the channel at the distance x is h(x), a width of the channel at the distance x is w(x), and a diameter of the megakaryocyte is x_{d}, w(x) is determined according to a frequency of a megakaryocyte having a diameter of h(x), and the channel is configured such that w(x) increases as a frequency of a megakaryocyte having a diameter x_{d} of h(x) increases.
